(19) United States
(12) Patent Application Publication
(10) Pub. No.: US 2022/0356511 A1
(43) Pub. Date: Nov. 10, 2022

(54) METHOD FOR DIAGNOSING CHRONIC EXPOSURE TO POLLUTION

(71) Applicant: L’OREAL, Paris (FR)

(72) Inventor: Cécile CLAVAUD, Aulnay-Sous-Bois (FR)

(21) Appl. No.: 17/621,295

(22) PCT Filed: Jun. 24, 2020
(86) PCT No.: PCT/EP2020/067712

§ 371 Date: Dec. 21, 2021

(30) Foreign Application Priority Data
Jun. 24, 2019 (FR) FR1906829

(51) Int. Cl. C12Q 1/689

(52) U.S. Cl. CPC C12Q 1/689; C12Q 2600/158; C12Q 2600/118

(57) ABSTRACT

The present invention concerns a method, in particular in vitro, of diagnosing a chronic exposure of a subject, in particular of the skin of a subject, to pollution, comprising a step (a) of determining, in a skin sample of the subject, the level of at least one marker chosen from the group consisting of bacteria of the species Corynebacterium lipophiloflavum, Corynebacterium durum, Corynebacterium propinquum, Kytococcus sedentarius, Brevibacterium sp., Haemophilus parainfluenzae, Actinomyces oris, Neisseria sp., Abiotrophia defectiva, Alicycliphilus sp., Micrococcus luteus, Caulobacter sp., Streptococcus mitis/oralis/pneumoniae and Staphylococcus epidermidis.

The present invention relates to the diagnosis of chronic exposures of a subject, in particular the skin of a subject, to pollution.

Certain urban areas are regularly subject to pollution peaks. The individual in their daily environment and particularly in urban areas, may be subjected to multiple attacks on keratin materials, and, notably the skin, by various air pollutants.

Among known pollutants, we will first mention exhaust gases that have become a major issue in large cities, heavy metals, but also fine particles containing polycyclic aromatic hydrocarbons such as benzo[a]pyrene or benzoanthracene.

These pollutants will notably cause, at the skin level, the deposition of particles on the surface of the epidermis, and among other consequences, may induce early aging of the skin marked by the presence of wrinkles and/or fine lines, of extended macules, lentigo simplex, redness and/or a dull and/or heterogeneous appearance of the complexion, but also imperfections at the level of the skin and its complexion.

It is therefore important to be able to demonstrate chronic exposure to pollution.

There is, therefore, an important need for methods of diagnosing aesthetic degradations of the skin, in particular those linked to pollution.

The present invention meets this need.

The present invention arises from the unexpected finding by the inventors that the relative abundance of certain bacterial species in skin samples of individuals correlated with the exposure to certain pollutants, in particular certain PAHs (Polycyclic Aromatic Hydrocarbons), evidenced by the detection of these pollutants in hair samples of the subjects studied.

The object of the present invention is, therefore, a method, in particular in vitro, of diagnosing a chronic exposure of a subject, in particular of the skin of a subject, to pollution, comprising a step (a) of determining, in a sample of the subject's skin, in particular a sample of the surface of the subject's skin, the level of at least one marker chosen from the group consisting of bacteria of the species Corynebacterium lipophiloflavum, Corynebacterium durum, Corynebacterium propinquum, Kytococcus sedentarius, Brevibacterium sp., Haemophilus parainfluenzae, Actinomyces oris, Neisseria sp., Abiotrophia defectiva, Alicycliphilus sp., Micrococcus luteus, Caulobacter sp., Streptococcus mitis/oralis/pneumoniae and Staphylococcus epidermidis.

### DETAILED DESCRIPTION OF THE INVENTION

### Marker

The marker used in the context of the invention is chosen from the group consisting of bacteria of the species Corynebacterium lipophiloflavum, Corynebacterium durum, Corynebacterium propinquum, Kytococcus sedentarius, Brevibacterium sp., Haemophilus parainfluenzae, Actinomyces oris, Neisseria sp., Abiotrophia defectiva, Alicycliphilus sp., Micrococcus luteus, Caulobacter sp., Streptococcus mitis/oralis/pneumoniae and Staphylococcus epidermidis.

By “bacteria of the species Corynebacterium lipophiloflavum”, is meant herein Gram-positive bacteria of the genus Corynebacterium, whose type strain is the strain DSM44291 described in Funke et al. (1997) FEMS Microbiol. Lett. 150: 219-224.

By “bacteria of the species Corynebacterium durum”, is meant herein Gram-positive bacteria of the genus Corynebacterium whose type strain is the strain DSM45333 described in Riegel et al. (1997) Int. J. Syst. Bacteriol. 47: 1107-1111.

By “bacteria of the species Corynebacterium propinquum” is meant herein Gram-positive bacteria of the genus Corynebacterium, the type strain of which is the strain DSM44285 described in Riegel et al. (1993) FEMS Microbiol. Lett. 113: 229-234.

By “bacteria of the species Kytococcus sedentarius” is meant herein Gram-positive bacteria of the genus Kytococcus, previously called Micrococcus sedentarius, whose type strain is the strain DSM20547 described in Stackebrandt et al. (1995) Int. J. Syst. Bacteriol. 45: 682-692.

By “bacteria of the species Brevibacterium sp.” is meant herein Gram-positive bacteria of the Actinomycetales order, the type species of which is Brevibacterium linens.

By “bacteria of the species Haemophilus parainfluenzae” is meant herein commensal bacteria of the genus Haemophilus whose type strain is the strain DSM8978 described in Rivers (1922) Johns Hopkins Hospital Bulletin 33: 429-431.

By “bacteria of the species Actinomyces oris” is meant herein Gram-positive bacteria of the genus Actinomyces whose type strain is the strain ATCC27044 described in Henssge et al. (2009) Int. J. Syst. Evol. Microbiol. 59: 509-516.

By “bacteria of the species Neisseria sp.” Is meant herein Gram negative bacteria of the order Neisseriales whose type species is Neisseria gonorrhoae.

By “bacteria of the species Abiotrophia defectiva” is meant herein Gram-positive bacteria of the genus Abiotrophia, the type strain of which is the strain DSM9849 described in Kawamura et al. (1995) Int. J. Syst. Bacteriol. 45: 798-803.

By “bacteria of the species Alicycliphilus sp.” is meant herein bacteria of the Burkholderiales order whose type species is Alicycliphilus denitrificans.

By “bacteria of the species Micrococcus luteus”, is meant herein Gram-positive, spherical, saprophytic bacteria of the family of Micrococcaceae whose type strain is the strain DSM20030 described in Wieser et al. (2002) Int. J. Syst. Evol. Microbiol. 52: 629-637.

By “bacteria of the species Caulobacter sp.” is meant herein bacteria of the order Caulobacterales whose type species is Caulobacter vibrioides.

By “bacteria of the species Streptococcus mitis/oralis/pneumoniae” is meant herein bacteria of the genus Streptococcus being of the species Streptococcus mitis (whose type strain is the strain NCTC3165 described in Andrewes et al. (1906) Lancet 2: 708-713), of the species Streptococcus oralis (whose type strain is the strain DSM20627 described in Bridge et al. (1982) Int. J. Syst. Bacteriol. 32: 410-415) or of the species Streptococcus pneumonia (whose type strain is the strain DSM20566 described in Klein (1884) Practitioner 32: 321-352).

By “bacteria of the species Staphylococcus epidermidis” is meant herein Gram-positive bacteria, facultative anaerobes, of the genus Staphylococcus whose type strain is the strain DSM20044 described in Evans (1916) J. Infect. Dis. 18: 437-476.

In a particular embodiment, said at least one marker is chosen from the group consisting of bacteria of the species Corynebacterium lipophiloflavum, Corynebacterium durum, Kytococcus sedentarius, Brevibacterium sp., Haemophilus parainfluenzae, Actinomyces oris, Neisseria sp., Abiotrophia defectiva, Alicycliphilus sp., Micrococcus luteus, Caulobacter sp. and Streptococcus mitis/oralis/pneumoniae.

In an alternative embodiment, said at least one marker is chosen from the group consisting of bacteria of the species Corynebacterium propinquum and Staphylococcus epidermidis.

### Diagnostic Method

The diagnostic method according to the invention is a method of diagnosing chronic exposure of a subject, in particular the skin of a subject, to pollution.

By “pollution” is meant here exposure to particles of matter, in particular to Polycyclic Aromatic Hydrocarbons (PAH).

Thus, in a particular embodiment, the pollution is PAH pollution.

As is well known to those skilled in the art, PAHs are a subfamily of aromatic hydrocarbons, the structure of which comprises at least 2 condensed aromatic rings. They are produced by the incomplete combustion of organic matter (coal, oil, gas, waste and biomass) and released as environmental contaminants.

PAHs typically include acenaphthylene, acenaphthene, fluorene, phenanthrene, anthracene, fluoranthene, pyrene, benzo[a]anthracene, chryzene, benzo-b-fluoranthene, benzo-k-fluoranthene, benzo[a]pyrene, indeno[1,2,3-cd]pyrene, dibenz-a-h-anthracene, benzo-g-h-i-perylene.

In a particular embodiment, the PAHs are selected from acenaphthylene, fluorene, phenanthrene, anthracene, fluoranthene, pyrene, benzo[a]anthracene and benzo[a]pyrene.

By “chronic exposure” is meant here a regular exposure, in particular daily exposure over a given period, preferably a period of more than a year, in particular a period of twelve months (typically corresponding to a growth of 12 cm in hair), possibly interrupted one or more times for short durations, typically durations of less than a month, preferably less than a week.

The diagnostic method according to the invention comprises a step (a) of determining, in a sample of the subject's skin, in particular a sample of the surface of the skin of a subject, the level of at least one marker chosen from the group consisting of bacteria as defined in the “Marker” section above.

In a particular embodiment, the level of said at least one marker in the sample is the relative abundance of said at least one marker in the sample.

By “relative abundance” is meant herein the relative amount as a percentage of a given taxon in relation to the total number of taxa in the sample.

The level of said at least one marker may be determined by any suitable technique.

In a particular embodiment, the level of said at least one marker is determined by sequencing, in particular typically when the level of said at least one marker is the relative abundance of said at least one marker in the sample, by metagenomic sequencing, especially of the shotgun type.

Typically, the bacterial genomic DNA present in the skin sample is extracted and then subjected to sequencing and quantification by metagenomics of the shotgun type.

In a particular embodiment, the diagnostic method according to the invention further comprises the steps consisting in:

(b) comparing the level of said at least one marker measured in step (a) with a control, and

(c) based on the comparison in step (b), determining whether the subject, in particular the subject's skin, underwent chronic exposure to pollution.

In a particular embodiment, the control is a reference value.

In a particular embodiment, the reference value is determined by the average value of the level of said marker in a determined population, for example a population in a determined age group, and/or having a defined skin type.

In a particular embodiment, the reference value is the average value of the level of said marker in a population of subjects, in particular subjects as defined below, living in a city with low pollution, in particular a city with an air quality index below 100 for less than 100 days, especially less than 85 days, over a year.

In a particular embodiment, said at least one marker is chosen from the group consisting of bacteria of the species Corynebacterium lipophiloflavum, Corynebacterium durum, Kytococcus sedentarius, Brevibacterium sp., Haemophilus parainfluenzae, Actinomyces oris, Neisseria sp., Abiotrophia defectiva, Alicycliphilus sp., Micrococcus luteus, Caulobacter sp. and Streptococcus mitis/oralis/pneumoniae, and the subject is diagnosed as having undergone chronic exposure to pollution if the level of said at least one marker measured in step (a), in particular the relative abundance of said at least one marker, is higher, in particular significantly higher, to a control,

the control level being typically the level of said marker, in particular the relative abundance of said marker, in a skin sample of a subject living in a city with little pollution, in particular a city with an air quality index of less than 100 for less than 100 days, in particular less than 85 days, over a year, and
the level of the markers being typically determined by sequencing, in particular by metagenomic sequencing of the shotgun type, typically as described above.

In another particular embodiment, said at least one marker is chosen from the group consisting of bacteria of the species Corynebacterium propinquum and Staphylococcus epidermidis, and the subject is diagnosed as having undergone chronic exposure to pollution if the level of said at least one marker measured in step (a), in particular the relative abundance of said at least one marker, is lower, in particular significantly lower, than a control,

the control level being typically the level of said marker, in particular the relative abundance of said marker, in a skin sample of a subject living in a city with little pollution, in particular a city with an air quality index of less than 100 for less than 100 days, in particular less than 85 days, over a year, and
the level of the markers being typically determined by sequencing, in particular by metagenomic sequencing of the shotgun type, typically as described above.

By “significantly lower” according to the invention is meant a statistically significant reduction in the level of the marker compared to the control level.

By “significantly higher” according to the invention is meant a statistically significant increase in the level of the marker compared to the control level.

In a particular embodiment, the pollution is a benzo[a]pyrene pollution, and said at least one marker is chosen from the group consisting of bacteria of the species Corynebacterium lipophiloflavum and Kytococcus sedentarius.

In this particular embodiment, the subject is diagnosed as having undergone chronic exposure to a benzo[a]pyrene pollution if the level of said at least one marker measured in step (a), in particular the relative abundance of said at least one marker, is higher, in particular significantly higher, to a control,

the control level being typically the level of said marker, in particular the relative abundance of said marker, in a skin sample of a subject living in a city with little pollution, in particular a city with an air quality index of less than 100 for less than 100 days, in particular less than 85 days, over a year, and
the level of the markers being typically determined by sequencing, in particular by metagenomic sequencing of the shotgun type, typically as described above.

In another particular embodiment, the pollution is a benzo[a]anthracene pollution, and said at least one marker is the bacteria of the species Corynebacterium durum.

In this particular embodiment, the subject is diagnosed as having undergone chronic exposure to benzo[a]anthracene pollution if the level of said at least one marker measured in step (a), in particular the relative abundance of said at least one marker, is higher, in particular significantly higher, to a control,

the control level being typically the level of said marker, in particular the relative abundance of said marker, in a skin sample of a subject living in a city with little pollution, in particular a city with an air quality index of less than 100 for less than 100 days, in particular less than 85 days, over a year, and
the level of the markers being typically determined by sequencing, in particular by metagenomic sequencing of the shotgun type, typically as described above.

In another particular embodiment, the pollution is a fluoranthene pollution, and said at least one marker is chosen from the group consisting of bacteria of the species Corynebacterium lipophiloflavum and Alicycliphilus sp.

In this particular embodiment, the subject is diagnosed as having undergone chronic exposure to fluoranthene pollution if the level of said at least one marker measured in step (a), in particular the relative abundance of said at least one marker, is higher, in particular significantly higher, to a control,

the control level being typically the level of said marker, in particular the relative abundance of said marker, in a skin sample of a subject living in a city with little pollution, in particular a city with an air quality index of less than 100 for less than 100 days, in particular less than 85 days, over a year, and
the level of the markers being typically determined by sequencing, in particular by metagenomic sequencing of the shotgun type, typically as described above.

In another particular embodiment, the pollution is a fluorene pollution, and said at least one marker is chosen from the group consisting of the species Corynebacterium lipophiloflavum, Kytococcus sedentarius, Brevibacterium sp., Haemophilus parainfluenzae, Actinomyces oris, Caulobacter sp. and Staphylococcus epidermidis.

In this particular embodiment, the subject is diagnosed as having undergone chronic exposure to fluorene pollution if
the level of the species Corynebacterium lipophiloflavum, Kytococcus sedentarius, Brevibacterium sp., Haemophilus parainfluenzae, Actinomyces oris and/or Caulobacter sp measured in step (a), in particular their relative abundance, is higher, in particular significantly higher, than a control, and/or
the level of the Staphylococcus epidermis species measured in step (a), in particular its relative abundance, is lower, in particular significantly lower, than a control

the control level being typically the level of said marker, in particular the relative abundance of said marker, in a skin sample of a subject living in a city with little pollution, in particular a city with an air quality index of less than 100 for less than 100 days, in particular less than 85 days, over a year, and

the level of the markers being typically determined by sequencing, in particular by metagenomic sequencing of the shotgun type, typically as described above.

In another particular embodiment, the pollution is an anthracene pollution, and said at least one marker is chosen from the group consisting of bacteria of the species Neisseria sp. and Abiotrophia defectiva.

In this particular embodiment, the subject is diagnosed as having undergone chronic exposure to anthracene pollution if the level of said at least one marker measured in step (a), in particular the relative abundance of said at least one marker, is higher, in particular significantly higher, to a control,

the control level being typically the level of said marker, in particular the relative abundance of said marker, in a skin sample of a subject living in a city with little pollution, in particular a city with an air quality index of less than 100 for less than 100 days, in particular less than 85 days, over a year, and
the level of the markers being typically determined by sequencing, in particular by metagenomic sequencing of the shotgun type, typically as described above.

In another particular embodiment, the pollution is a pyrene pollution, and said at least one marker is chosen from the group consisting of the species Kytococcus sedentarius, Brevibacterium sp., Haemophilus parainfluenzae and Staphylococcus epidermidis.

In this particular embodiment, the subject is diagnosed as having undergone chronic exposure to pyrene pollution if
the level of the species Kytococcus sedentarius, Brevibacterium sp. and/or Haemophilus parainfluenzae measured in step (a), in particular their relative abundance, is higher, in particular significantly higher, than a control, and/or
the level of the Staphylococcus epidermis species measured in step (a) is lower, in particular significantly lower, than a control
   the control level being typically the level of said marker, in particular the relative abundance of said marker, in a skin sample of a subject living in a city with little pollution, in particular a city with an air quality index of less than 100 for less than 100 days, in particular less than 85 days, over a year, and
   the level of the markers being typically determined by sequencing, in particular by metagenomic sequencing of the shotgun type, typically as described above.

In another particular embodiment, the pollution is an acenaphthylene pollution, and said at least one marker is chosen from the group consisting of the species Alicycliphilus sp, Actinomyces oris, and Staphylococcus epidermidis.

In this particular embodiment, the subject is diagnosed as having undergone chronic exposure to acenaphthylene pollution if
the level of Alicycliphilus sp. and/or Actinomyces oris measured in step (a), in particular their relative abundance, is higher, in particular significantly higher, than a control, and/or
the level of the Staphylococcus epidermis species measured in step (a), in particular its relative abundance, is lower, in particular significantly lower, than a control
   the control level being typically the level of said marker, in particular the relative abundance of said marker, in a skin sample of a subject living in a city with little pollution, in particular a city with an air quality index of less than 100 for less than 100 days, in particular less than 85 days, over a year, and
   the level of the markers being typically determined by sequencing, in particular by metagenomic sequencing of the shotgun type, typically as described above.

In another particular embodiment, the pollution is phenanthrene pollution, and said at least one marker is chosen from the group consisting of the species Corynebacterium durum, Kytococcus sedentarius, Micrococcus luteus, Streptococcus mitis/oralis/pneumoniae and Corynebacterium propinquum.

In this particular embodiment, the subject is diagnosed as having undergone chronic exposure to phenanthrene pollution if
the level of the species Corynebacterium durum, Kytococcus sedentarius, Micrococcus luteus and/or Streptococcus mitis/oralis/pneumoniae measured in step (a), in particular their relative abundance, is higher, in particular significantly higher, than a control, and/or
the level of the species Corynebacterium propinquum measured in step (a), in particular its relative abundance, is lower, in particular significantly lower, than a control
   the control level being typically the level of said marker, in particular the relative abundance of said marker, in a skin sample of a subject living in a city with little pollution, in particular a city with an air quality index of less than 100 for less than 100 days, in particular less than 85 days, over a year, and
   the level of the markers being typically determined by sequencing, in particular by metagenomic sequencing of the shotgun type, typically as described above.

In another particular embodiment, said at least one marker is bacteria of the species Corynebacterium lipoflavum and the pollution is pollution with benzo[a]pyrene, fluoranthene and/or fluorene.

In this particular embodiment, the subject is diagnosed as having suffered chronic exposure to pollution with benzo[a]pyrene, fluoranthene and/or fluorene if the level of bacteria of the species Corynebacterium lipoflavum measured in step (a), in particular their relative abundance, is higher, in particular significantly higher, than a control,

the control level being typically the level of said marker, in particular the relative abundance of said marker, in a skin sample of a subject living in a city with little pollution, in particular a city with an air quality index of less than 100 for less than 100 days, in particular less than 85 days, over a year, and
the level of the markers being typically determined by sequencing, in particular by metagenomic sequencing of the shotgun type, typically as described above.

In another particular embodiment, said at least one marker is bacteria of the species Corynebacterium durum and the pollution is pollution with benzo[a]pyrene, and/or phenanthrene.

In this particular embodiment, the subject is diagnosed as having undergone chronic exposure to pollution with benzo[a]pyrene, and/or phenanthrene if the level of bacteria of the species Corynebacterium durum measured in step (a), in particular their relative abundance, is higher, in particular significantly higher, than a control,

the control level being typically the level of said marker, in particular the relative abundance of said marker, in a skin sample of a subject living in a city with little pollution, in particular a city with an air quality index of less than 100 for less than 100 days, in particular less than 85 days, over a year, and
the level of the markers being typically determined by sequencing, in particular by metagenomic sequencing of the shotgun type, typically as described above.

In another particular embodiment, said at least one marker is bacteria of the species Kytococcus sedentarius and the pollution is pollution with benzo[a]pyrene, fluorene, pyrene and/or phenanthrene.

In this particular embodiment, the subject is diagnosed as having undergone chronic exposure to pollution with benzo[a]pyrene, fluorene, pyrene and/or phenanthrene if the level of bacteria of the species Kytococcus sedentarius measured at step (a), in particular their relative abundance, is higher, in particular significantly higher, than a control,

the control level being typically the level of said marker, in particular the relative abundance of said marker, in a skin sample of a subject living in a city with little pollution, in particular a city with an air quality index of less than 100 for less than 100 days, in particular less than 85 days, over a year, and
the level of the markers being typically determined by sequencing, in particular by metagenomic sequencing of the shotgun type, typically as described above.

In another particular embodiment, said at least one marker is bacteria of the species Brevibacterium sp. and the pollution is fluorene and/or pyrene pollution.

In this particular embodiment, the subject is diagnosed as having undergone chronic exposure to fluorene and/or pyrene pollution if the level of bacteria of the species Brevibacterium sp. measured in step (a), in particular their relative abundance, is higher, in particular significantly higher, than a control,

the control level being typically the level of said marker, in particular the relative abundance of said marker, in a skin sample of a subject living in a city with little pollution, in particular a city with an air quality index of less than 100 for less than 100 days, in particular less than 85 days, over a year, and
the level of the markers being typically determined by sequencing, in particular by metagenomic sequencing of the shotgun type, typically as described above.

In another particular embodiment, said at least one marker is bacteria of the species Haemophilus parainfluenza and the pollution is pollution with fluorene and/or pyrene.

In this particular embodiment, the subject is diagnosed as having undergone chronic exposure to fluorene and/or pyrene pollution if the level of bacteria of the Haemophilus parainfluenza species measured in step (a), in particular their relative abundance, is higher, in particular significantly higher, than a control,

the control level being typically the level of said marker, in particular the relative abundance of said marker, in a skin sample of a subject living in a city with little pollution, in particular a city with an air quality index of less than 100 for less than 100 days, in particular less than 85 days, over a year, and
the level of the marker being typically determined by sequencing, in particular by metagenomic sequencing of the shotgun type, typically as described above.

In another particular embodiment, said at least one marker is bacteria of the species Actinomyces oris and the pollution is pollution with fluorene and/or acenaphthylene.

In this particular embodiment, the subject is diagnosed as having undergone chronic exposure to pollution with fluorene and/or acenaphthylene if the level of bacteria of the species Actinomyces oris measured in step (a), in particular their relative abundance, is higher, in particular significantly higher, than a control,

the control level being typically the level of said marker, in particular the relative abundance of said marker, in a skin sample of a subject living in a city with little pollution, in particular a city with an air quality index of less than 100 for less than 100 days, in particular less than 85 days, over a year, and
the level of the markers being typically determined by sequencing, in particular by metagenomic sequencing of the shotgun type, typically as described above.

In another particular embodiment, said at least one marker is bacteria of the species Neisseria sp. and the pollution is anthracene pollution.

In this particular embodiment, the subject is diagnosed as having undergone chronic exposure to anthracene pollution if the level of bacteria of the species Neisseria sp. measured in step (a), in particular their relative abundance, is higher, in particular significantly higher, than a control,

the control level being typically the level of said marker, in particular the relative abundance of said marker, in a skin sample of a subject living in a city with little pollution, in particular a city with an air quality index of less than 100 for less than 100 days, in particular less than 85 days, over a year, and
the level of the markers being typically determined by sequencing, in particular by metagenomic sequencing of the shotgun type, typically as described above.

In another particular embodiment, said at least one marker is bacteria of the species Abiotrophia defectiva and the pollution is anthracene pollution.

In this particular embodiment, the subject is diagnosed as having undergone chronic exposure to anthracene pollution if the level of bacteria of the species Abiotrophia defectiva measured in step (a), in particular their relative abundance, is higher, in particular significantly higher, to a control,

the control level being typically the level of said marker, in particular the relative abundance of said marker, in a skin sample of a subject living in a city with little pollution, in particular a city with an air quality index of less than 100 for less than 100 days, in particular less than 85 days, over a year, and
the level of the markers being typically determined by sequencing, in particular by metagenomic sequencing of the shotgun type, typically as described above.

In another particular embodiment, said at least one marker is bacteria of the species Alicycliphilus sp. and the pollution is pollution with fluoranthene and/or acenaphthylene.

In this particular embodiment, the subject is diagnosed as having undergone chronic exposure to pollution with fluoranthene and/or acenaphthylene if the level of bacteria of the species Alicycliphilus sp. measured in step (a), in particular their relative abundance, is higher, in particular significantly higher, than a control,

the control level being typically the level of said marker, in particular the relative abundance of said marker, in a skin sample of a subject living in a city with little pollution, in particular a city with an air quality index of less than 100 for less than 100 days, in particular less than 85 days, over a year, and
the level of the markers being typically determined by sequencing, in particular by metagenomic sequencing of the shotgun type, typically as described above.

In another particular embodiment, said at least one marker is bacteria of the Micrococcus luteus species and the pollution is phenanthrene pollution.

In this particular embodiment, the subject is diagnosed as having undergone chronic exposure to phenanthrene pollution if the level of bacteria of the species Micrococcus luteus measured in step (a), in particular their relative abundance, is higher, in particular significantly higher, to a control,

the control level being typically the level of said marker, in particular the relative abundance of said marker, in a skin sample of a subject living in a city with little pollution, in particular a city with an air quality index of less than 100 for less than 100 days, in particular less than 85 days, over a year, and
the level of the markers being typically determined by sequencing, in particular by metagenomic sequencing of the shotgun type, typically as described above.

In another particular embodiment, said at least one marker is bacteria of the species Caulobacter sp. and the pollution is fluorene pollution.

In this particular embodiment, the subject is diagnosed as having undergone chronic exposure to fluorene pollution if the level of bacteria of the species Caulobacter sp. measured in step (a), in particular their relative abundance, is higher, in particular significantly higher, than a control,

the control level being typically the level of said marker, in particular the relative abundance of said marker, in a skin sample of a subject living in a city with little pollution, in particular a city with an air quality index of less than 100 for less than 100 days, in particular less than 85 days, over a year, and
the level of the markers being typically determined by sequencing, in particular by metagenomic sequencing of the shotgun type, typically as described above.

In another particular embodiment, said at least one marker is bacteria of the species Streptococcus mitis/oralis/pneumoniae and the pollution is pollution with phenanthrene.

In this particular embodiment, the subject is diagnosed as having undergone chronic exposure to phenanthrene pollution if the level of bacteria of the species Streptococcus mitis/oralis/pneumoniae measured in step (a), in particular their relative abundance, is higher, in particular significantly higher, than a control,

the control level being typically the level of said marker, in particular the relative abundance of said marker, in a skin sample of a subject living in a city with little pollution, in particular a city with an air quality index of less than 100 for less than 100 days, in particular less than 85 days, over a year, and
the level of the marker being typically determined by sequencing, in particular by metagenomic sequencing of the shotgun type, typically as described above.

In another particular embodiment, said at least one marker is bacteria of the species Corynebacterium propinquum and the pollution is phenanthrene pollution.

In this particular embodiment, the subject is diagnosed as having undergone chronic exposure to phenanthrene pollution if the level of bacteria of the species Corynebacterium propinquum measured in step (a), in particular their relative abundance, is lower, in particular significantly lower, than a control,

the control level being typically the level of said marker, in particular the relative abundance of said marker, in a skin sample of a subject living in a city with little pollution, in particular a city with an air quality index of less than 100 for less than 100 days, in particular less than 85 days, over a year, and
the level of the marker being typically determined by sequencing, in particular by metagenomic sequencing of the shotgun type, typically as described above.

In another particular embodiment, said at least one marker is bacteria of the species Staphylococcus epidermis and the pollution is pollution with fluorene, pyrene and/or acenaphthylene.

In this particular embodiment, the subject is diagnosed as having undergone chronic exposure to pollution with fluorene, pyrene and/or acenaphthylene if the level of bacteria of the species Staphylococcus epidermis measured in step (a), in particular their relative abundance, is lower, in particular significantly lower, than a control,

the control level being typically the level of said marker, in particular the relative abundance of said marker, in a skin sample of a subject living in a city with little pollution, in particular a city with an air quality index of less than 100 for less than 100 days, in particular less than 85 days, over a year, and
the level of the marker being typically determined by sequencing, in particular by metagenomic sequencing of the shotgun type, typically as described above.

According to a preferred embodiment, the skin sample of the subject used in the diagnostic method according to the invention is a sample taken, preferably in a non-invasive manner, from the skin of the subject, preferably from the face and/or the head of the subject, particularly on the subject's cheek and/or scalp. Thus, in one embodiment, the skin sample is a cheek skin or scalp sample. Preferably, the skin sample comes from the stratum corneum, in particular from the surface of the stratum corneum.

The stratum corneum is the layer furthest from the epidermis, and includes the surface of the skin. It is mainly composed of dead cells.

According to an embodiment, the diagnostic method according to the invention comprises a step of taking the subject's skin sample, in particular from the subject's skin surface. This step is preferably carried out in a non-invasive manner, and in particular does not require local anesthesia. According to a preferred embodiment, the step of taking the sample is carried out by rubbing the surface of the skin, in particular with a wet cotton swab.

In a particular embodiment, the skin sample is thus taken by rubbing the surface of the skin.

By “subject” is meant herein a human being, preferably aged 25 to 45 years. Preferably, the subject is a woman. Preferably, the subject is of Asian type.

In a particular embodiment, the subject is between 25 and 45 years old.

Chronic exposure to pollution may typically lead to early aging of the skin marked by the presence of wrinkles and/or fine lines, extended macules, lentigo simplex, redness and/or a dull and/or heterogeneous appearance of the complexion, but also aesthetic degradations of the skin such as pigmentary disorders.

Therefore, the present invention relates to a method of preventing early aging of the skin and/or aesthetic degradations of the skin, induced by chronic exposure to pollution, in a subject, said method comprising the following steps:
A) diagnosing the subject as undergoing chronic exposure to pollution, by implementing the diagnostic method according to the invention,
B) if the subject is diagnosed as undergoing chronic exposure to pollution, treating said subject with a cosmetic composition enabling slowing down early aging of the skin and/or aesthetic degradations of the skin.

In a particular embodiment, the cosmetic composition used in the treatment method of the invention comprises probiotics and/or prebiotics, in particular making it possible to promote the presence of the commensal flora.

The present invention is described in more detail through the examples below.

### Example

The example below shows the identification of a signature of 14 bacterial species which are significantly modulated in skin samples from individuals exposed to chronic pollution (based on the detection of high levels of pollutants in samples of their hair).

### Materials and Methods

Main Steps:
skin sampling
global metagenomics known as “shotgun”
analysis of the correlation between PAH and bacterial species

### Description of Subjects

All subjects from each city visited the facilities in Baoding and Dalian (China). Cheek skin samples were collected from 204 healthy Chinese women, aged between 25 and 45, 102 of whom lived in the relatively rural and industrial city of Baoding, a city in northern China's province of Hebei recording high levels of air pollution (about 90 μg of PM_{2.5}/m³ of air), and 102 of whom lived in Dalian, a city in the north of China, urbanized and modern in the province of Liaoning with a lower degree of recorded air pollution (approximately 30 μg of PM_{2.5}/m³ of air). These cities are located at the same latitude, and have shared a similar climate and equivalent UV exposure (UV index) for the past 15 years.

Participants living in both cities were assessed for their exposure to PAHs (Polycyclic Aromatic Hydrocarbons) in 12 cm hair samples (reflecting the extent of exposure for a period of one year) (Palazzi et al. (2018) Env. Int. 121: 1341-1354). Specifically, at Baoding, the median concentration was 1.5 to 2.8 times higher for the parent PAHs, and 1.1 to 2.3 times higher for the PAH metabolites than that of Dalian. Among the quantified parent PAHs, higher levels were observed for phenanthrene, fluoranthene, pyrene, fluorene, acenaphthylene and anthracene, while for PAH metabolites, the levels of 9-OH fluorene, of 2-OH-naphthalene and 1-OH-anthracene were higher (Palazzi et al. (2018) Env. Int. 121: 1341-1354).

### Subjects and Sample Collection

None of the participants received any systemic antibiotics or antifungals one month before sampling, had a severe skin disorder, or had used skin or systemic depigmentation/bleaching treatments for three months before sampling, or an exfoliating product for one month before sampling. They were asked to wash their faces using the neutral soap provided without antibacterial compounds for 3 days (once a day) before sampling. The last shampoo and soap were applied respectively 48 and 24 hours before sampling. No other products were allowed on the scalp, hair and face until the sampling was completed.

The microbiota sampling was carried out in a controlled atmosphere at 22° C. and 60% humidity. Samples for microbiome analysis were collected using sterile dry cotton swabs which were heated to 150° C. and pre-wetted with ST solution (0.15 M NaCl with 0.1% Tween 20). For cheek samples, the swabs were soaked in a collection buffer and rubbed firmly on the cheek for 60 seconds to cover an area of 1 cm×2 cm. For the scalp samples, the swabs were rubbed firmly on the scalp surface along one line in 4 passes, then the swab was moved to another line using a comb. These steps were carried out 6 times to cover a total area of 4 cm². After sampling, each cotton swab was placed in a microtube and immediately frozen in liquid nitrogen, and stored at −80° C. before extraction of genomic DNA (gDNA).

### Preparation of the Metagenomic Sequencing Library and Analysis

Sixteen samples (9 from the cheeks and 7 from the scalp) were selected for analysis by metagenomic sequencing. The samples were selected based on extreme levels (low versus high) of PAH exposure of individuals in the two cities.
These samples are shown in Table 1 below.

TABLE 1
Level of PAHs determined in the individuals studied

| Individual | 8 | 8 | 12 | 12 | 14 | 14 | 26 | 26 |
|---|---|---|---|---|---|---|---|---|
| Site | J | S | J | S | J | S | J | S |
| City | B | B | B | B | B | B | B | B |
| Age group | 30-34 | 30-34 | 35-39 | 35-39 | 40-45 | 40-45 | 40-45 | 40-45 |
| Age | 30 | 30 | 36 | 36 | 40 | 40 | 40 | 40 |
| Acenaphthylene | 8.3* | 8.3* | 13.65* | 13.65* | 11.4* | 11.4* | 15.49* | 15.49* |
| Fluorene | 15.75* | 15.75* | ND | ND | 86.62* | 86.62* | 18.48* | 18.48* |
| Phenanthrene | 36.37* | 36.37* | ND | ND | 110.91 | 110.91 | 24.11* | 24.11* |
| Anthracene | 285.79 | 285.79 | 183.57 | 183.57 | 888.8 | 888.8 | 263.63 | 263.63 |
| Fluoranthene | 18.65 | 18.65 | 18.41 | 18.41 | 40.94 | 40.94 | 20.62 | 20.62 |
| Pyrene | 88.65 | 88.65 | 67.42 | 67.42 | 203.6 | 203.6 | 65.56 | 65.56 |
| Acenaphthylene | 66.38 | 66.38 | 88.42 | 88.42 | 123.73 | 123.73 | 47.78 | 47.78 |
| Benzo[a]anthracene | 4.75 | 4.75 | 6.04 | 6.04 | 8.22 | 8.22 | 3.53 | 3.53 |
| Benzo[b]fluoranthene | 3.71 | 3.71 | 3.15 | 3.15 | 4.59 | 4.59 | 2.22 | 2.22 |
| Benzo[a]pyrene | 1.63 | 1.63 | 1.39 | 1.39 | 2.38 | 2.38 | 1.4 | 1.4 |
| Individual | 59 | 59 | 446 | 446 | 482 | 501 | 501 | 518 |
| Site | J | S | J | S | J | S | J | J |
| City | B | B | D | D | D | D | D | D |
| Age group | 25-29 | 25-29 | 30-34 | 30-34 | 30-34 | 40-45 | 40-45 | 25-29 |
| Age | 28 | 28 | 33 | 33 | 30 | 45 | 45 | 29 |
| Acenaphthylene | 10.71* | 10.71* | 7.32* | 7.32* | ND | 8.26* | 8.26* | ND |
| Fluorene | ND | ND | 8.53* | 8.53* | ND | ND | ND | ND |
| Phenanthrene | 22.18* | 22.18* | 4.87* | 4.87* | ND | ND | ND | ND |
| Anthracene | 160.64 | 160.64 | 26.51* | 26.51* | 12.43* | 5.94* | 5.94* | ND |
| Fluoranthene | 9.55 | 9.55 | ND | ND | ND | ND | ND | ND |
| Pyrene | 65.87 | 65.87 | 1.77* | 1.77* | ND | ND | ND | ND |
| Acenaphthylene | 42.91 | 42.91 | ND | ND | 10.35 | 4.62* | 4.62* | 10.86 |
| Benzo[a]anthracene | 3.57 | 3.57 | 0.07* | 0.07* | 0.56* | 0.04* | 0.04* | 0.11* |
| Benzo[b]fluoranthene | 3.6 | 3.6 | 0.12* | 0.12* | 0.23* | 0.26* | 0.26* | 0.14* |
| Benzo[a]pyrene | 1.66 | 1.66 | ND | ND | ND | ND | ND | ND |

J: cheek;
S: scalp;
B: Baoding;
D: Dalian
The levels are presented in pg/mg of hair.
The limit of quantification (LOQ) for the various compounds was as follows: acenaphthylene: 20 pg/mg; acenaphthene: 100 pg/mg; fluorene: 100 pg/mg; phenanthrene: 100 pg/mg; anthracene: 5 pg/mg; fluoranthene: 10 pg/mg; pyrene: 10 pg/mg; benzo[a]anthracene: 1 pg/mg; benzo[b]fluoranthene: 1 pg/mg; benzo[a]pyrene: 1 pg/mg.
The measurements that are under the LOQ for each PAH (indicated by *) were used for the analysis.
ND: not detected. To perform the analyzes, the ND measurements were converted to the minimum value present in the data for this PAH, divided by the square root of 2.

The gDNA was extracted using the PowerSoil DNA® isolation kit (MO BIO Laboratories, Carlsbad, Calif., USA) following the manufacturer's instructions with the modifications described in Leung et al. (2014) Appl. About. Microbiol. 80: 6760-6770. In addition, following the C6 elution, the eluate was passed through the same column filter once more to increase the yield. Negative DNA-free water controls were extracted in parallel.

The gDNA of each sample was normalized to 10 ng/μl before preparation of the library. Library preparation was performed using the standard Illumina protocol, and the final library was quantified with the TapeStation 2200® (Agilent, Santa Clara, Calif., USA) and sequenced with the Illumina MiSeq® platform by SeqMatic LLC (Fremont, Calif., USA) to generate 150 bp paired end readings.

Raw readings were subjected to quality control and disposal of human DNA readings using KneadData® v0.6.1 (http://huttenhower.sph.harvard.edu/kneaddata), readings shorter than 50 bp and the readings corresponding to the human reference genome hg38 being eliminated. Taxonomic alignments were made on filtered readings using MetaPhlAn2® v2.6.0 (Truong et al. (2015) Nat. Methods 12: 902-903). Functional allocations were performed using HUMAnN2® v0.11.1, with output results expressed in reading per kilobase per million readings (RPKM). The HUMAnN2 results were converted into “KEGG Orthology” (KO) gene families for a “heatmap” representation and a multivariate analysis of functional genes (Abubucker et al. (2012) PLoS Comput. Biol. 8: e1002358). MaAsLin was performed to associate exposure to PAHs with MetaPh1An2 and HUMAnN2 data, an FDR-corrected p-value (q-value) less than or equal to 0.25 being considered significant as shown in Lim et al. (2016) Sci. Rep. 6: 23745, or Knights et al. (2014) Genome Med. 6:107.

### Statistical Analysis

The non-parametric Mann-Whitney test was performed to test the significance between the 2 groups, while the Kruskal-Wallis test was performed to test the significance between 3 or more groups. An FDR correction was performed using the “p.adjust” command in R.

### Results

Table 2 below presents the main correlations identified between a change in PAH of −40 units and changes in percentage of relative abundance (RA) of significant species (q-value≤0.25).

TABLE 2
Main correlations identified between a change in PAH of −40 units and
the changes in percentage of relative abundance (RA) of significant species.

| | | | Increase in | Change in % |
|---|---|---|---|---|
| | | Regression | PAH | of the |
| PAH | Species | coefficient | (hypothetical) | species RA |
| Benzo[a]pyrene | Corynebacterium lipophiloflavum | 2.3200 | 40 units | 92.8 |
| Benzo[a]pyrene | Kytococcus sedentarius | 1.0400 | 40 units | 41.6 |
| Benzo[a]anthracene | Corynebacterium durum | 0.2180 | 40 units | 8.72 |
| Fluoranthene | Corynebacterium lipophiloflavum | 0.1950 | 40 units | 7.8 |
| Fluorene | Brevibacterium sp, | 0.1580 | 40 units | 6.32 |
| Fluorene | Haemophilus parainfluenzae | 0.1540 | 40 units | 6.16 |
| Fluorene | Actinomyces oris | 0.1170 | 40 units | 4.68 |
| Anthracene | Neisseria sp, | 0.1040 | 40 units | 4.16 |
| Pyrene | Brevibacterium sp, | 0.0786 | 40 units | 3.144 |
| Pyrene | Haemophilus parainfluenzae | 0.0769 | 40 units | 3.076 |
| Fluorene | Kytococcus sedentarius | 0.0512 | 40 units | 2.048 |
| Pyrene | Actinomyces oris | 0.0449 | 40 units | 1.796 |
| Anthracene | Abiotrophia defectiva | 0.0442 | 40 units | 1.768 |
| Acenaphthylene | Alicycliphilus sp, | 0.0390 | 40 units | 1.56 |
| Acenaphthylene | Actinomyces oris | 0.0314 | 40 units | 1.256 |
| Pyrene | Kytococcus sedentarius | 0.0292 | 40 units | 1.168 |
| Fluorene | Corynebacterium lipophiloflavum | 0.0290 | 40 units | 1.16 |
| Fluoranthene | Alicycliphilus sp, | 0.0273 | 40 units | 1.092 |
| Phenanthrene | Micrococcus luteus | 0.0118 | 40 units | 0.472 |
| Fluorene | Caulobacter sp, | 0.0089 | 40 units | 0.3568 |
| Phenanthrene | Streptococcus | 0.0025 | 40 units | 0.1008 |
| | mitis/oralis/pneumoniae | | | |
| Phenanthrene | Corynebacterium durum | 0.0015 | 40 units | 0.06 |
| Phenanthrene | Kytococcus sedentarius | 0.0009 | 40 units | 0.03524 |
| Phenanthrene | Corynebacterium propinquum | −0.0019 | 40 units | −0.0752 |
| Pyrene | Staphylococcus epidermidis | −0.0320 | 40 units | −1.28 |
| Acenaphthylene | Staphylococcus epidermidis | −0.0448 | 40 units | −1.792 |
| Fluorene | Staphylococcus epidermidis | −0.0616 | 40 units | −2.464 |

The correlation for most PAH-species pairs was extrapolated based on changes in PAH of smaller magnitudes (i.e. 1.2 units).

In conclusion, a positive correlation between the percentage of RA of the species and an increase in PAH was observed for the following species:
Abiotrophia defectiva,
Actinomyces oris,
Alicycliphilus sp.,
Brevibacterium sp.,
Caulobacter sp.,
Corynebacterium durum,
Corynebacterium lipophiloflavum,
Haemophilus influenzae,
Kytococcus sedentarius,
Microccus luteus,
Neisseria sp., and
Streptococcus mitis/oralis/pneumoniae,
   and a negative correlation between the percentage of RA of the species and an increase in PAH was observed for the following species:
Staphylococcus epidermis, and
Corynebacterium propinquum.

1. A diagnostic method for diagnosing a chronic exposure of a the skin of a subject, to pollution, comprising a step (a) of determining, in a sample of the subject's skin, the level of at least one marker chosen from the group consisting of bacteria of the species Corynebacterium lipophiloflavum, Corynebacterium durum, Corynebacterium propinquum, Kytococcus sedentarius, Brevibacterium sp., Haemophilus parainfluenzae, Actinomyces oris, Neisseria sp., Abiotrophia defectivaus, Alicyclip. sp., Streptococcus mitis oralis/pneumoniae and Staphylococcus epidermidis.

2. The diagnostic method according to claim 1, wherein said at least one marker is chosen from the group consisting of bacteria of the species Corynebacterium lipophiloflavum, Corynebacterium durum, Kytococcus sedentarius, Brevibacterium sp., Haemophilus parainfluenzae, Actinomyces oris, Neisseria sp., Abiotrophia defectiva, Alicycliphilus sp., Micrococcus luteus, Caulobacter sp. and Streptococcus mitis oralis/pneumoniae.

3. The diagnostic method according to claim 1, wherein said at least one marker is chosen from the group consisting of bacteria of the species Corynebacterium propinquum and Staphylococcus epidermidis.

4. The diagnostic method according to claim 1, the method further comprising the steps consisting in:
(b) comparing the level of said at least one marker measured in step (a) with a control, and
(c) based on the comparison in step (b), determining whether the skin of the subject, has undergone chronic exposure to pollution.

5. The diagnostic method according to claim 4, wherein said at least one marker is chosen from the group consisting of bacteria of the species Corynebacterium lipophiloflavum, Corynebacterium durum, Kytococcus sedentarius, Brevibacterium sp., Haemophilus parainfluenzae, Actinomyces oris, Neisseria sp., Abiotrophia defectiva, Alicycliphilus sp., Micrococcus luteus, Caulobacter sp. and Streptococcus mitis oralis/pneumoniae, and the subject is diagnosed as having undergone chronic exposure to pollution if the level of said at least one marker measured in step (a) is higher than a control.

6. The diagnostic method according to claim 4, wherein said at least one marker is chosen from the group consisting of bacteria of the species Corynebacterium propinquum and Staphylococcus epidermidis, and the subject is diagnosed as having undergone chronic exposure to pollution, if the level of said at least one marker measured in step (a) is lower than a control.

7. The diagnostic method according to claim 1, wherein the level of said at least one marker is determined by metagenomic sequencing.

8. The diagnostic method according to claim 1, wherein the skin sample is a cheek skin or scalp sample.

9. The diagnostic method according to claim 1, wherein the pollution is pollution with polycyclic aromatic hydrocarbons (PAHs).

10. The diagnostic method according to claim 1, wherein the subject is between 25 and 45 years old.

11. The diagnostic method according to claim 2, the method further comprising the steps consisting in:
(b) comparing the level of said at least one marker measured in step (a) with a control, and
(c) based on the comparison in step (b), determining whether the skin of the subject, has undergone chronic exposure to pollution.

12. The diagnostic method according to claim 3, the method further comprising the steps consisting in:
(b) comparing the level of said at least one marker measured in step (a) with a control, and
(c) based on the comparison in step (b), determining whether the skin of the subject, has undergone chronic exposure to pollution.

13. The diagnostic_method according to claim 2, wherein the level of said at least one marker is determined by metagenomic sequencing.

14. The diagnostic_method according to claim 3, wherein the level of said at least one marker is determined by metagenomic sequencing.

15. The diagnostic_method according to claim 4, wherein the level of said at least one marker is determined by metagenomic sequencing.

16. The diagnostic_method according to claim 5, wherein the level of said at least one marker is determined by metagenomic sequencing.

17. The diagnostic_method according to claim 6, wherein the level of said at least one marker is determined by metagenomic sequencing.

18. The diagnostic method according to claim 2, wherein the skin sample is a cheek skin or scalp sample.

19. The diagnostic method according to claim 3, wherein the skin sample is a cheek skin or scalp sample.

20. The diagnostic method according to claim 4, wherein the skin sample is a cheek skin or scalp sample.
